(19) United States
(12) Patent Application Publication
(10) Pub. No.: US 2022/0361780 A1
(43) Pub. Date: Nov. 17, 2022

(54) GLUCOSE PREDICTION SYSTEMS AND ASSOCIATED METHODS

(71) Applicants: THE TEXAS A&M UNIVERSITY SYSTEM, College Station, TX (US); BAYLOR COLLEGE OF MEDICINE, Houston, TX (US)

(72) Inventors: Madhav Erraguntla, College Station, TX (US); Darpit K. Dave, College Station, TX (US); Balakrishna Haridas, College Station, TX (US); Daniel J. DeSalvo, Houston, TX (US); Siripoom V. McKay, Houston, TX (US); Mark Alan Lawley, College Station, TX (US); Achu Geetha Byju, College Station, TX (US)

(73) Assignees: THE TEXAS A&M UNIVERSITY SYSTEM, College Station, TX (US); BAYLOR COLLEGE OF MEDICINE, Houston, TX (US)

(21) Appl. No.: 17/769,883

(22) PCT Filed: Oct. 16, 2020
(86) PCT No.: PCT/US2020/056109

§ 371 Date: Apr. 18, 2022

Related U.S. Application Data
(60) Provisional application No. 62/923,104, filed on Oct. 18, 2019.

(51) Int. Cl. A61B 5/145; A61B 5/00

(52) U.S. Cl. CPC A61B 5/14532; A61B 5/7275; A61B 5/7282

(57) ABSTRACT

A method for predicting a hypoglycemic event or a hyperglycemic event includes (a) collecting one or more data streams, wherein at least one of the one or more data streams includes a glucose (GL) data stream corresponding to a user, (b) determining one or more features based on the one or more collected data streams, wherein at least one of the one or more features is distinct from each of the one or more data streams, (c) generating a prediction of whether or not a hypoglycemic event or a hyperglycemic event will occur by a prediction model based on the one or more features determined at (b), and (d) issuing an alert to the user in response to the prediction generated at (c) including a prediction that a hypoglycemic event or a hyperglycemic event will occur.

### CROSS-REFERENCE TO RELATED APPLICATIONS

This application claims benefit of U.S. provisional application Ser. No. 62/923,104 filed Oct. 18, 2019, and entitled “Machine Learning Based Glucose Prediction Algorithm,” which is hereby incorporated herein by reference in its entirety.

### STATEMENT REGARDING FEDERALLY SPONSORED RESEARCH OR DEVELOPMENT

This invention was made with government support under 1P50FD006428-01 awarded by the DHHS-Food and Drug Administration. The government has certain rights in this invention.

### BACKGROUND

Insulin is a hormone secreted by the pancreas to enable cells to capture blood sugar and help provide energy to the body. People suffering from diabetes may not use insulin properly (Type 2 diabetes) or may be unable to produce the required insulin (Type 1 diabetes). Type 1 diabetes, which may require rigorous day-to-day management, pivots around managing glucose (GL) levels through regular insulin delivery (e.g., through an insulin pump attached to the body or shots), diet, and a proper lifestyle.

Low GL levels (hypoglycemia) may have serious and acute health risks such as confusion, anxiety, loss of consciousness, seizures, and in extreme cases, coma and death. Due to the potentially serious and acute sequelae of hypoglycemia, many diabetic patients may tend to maintain higher than ideal GL levels resulting in long-term complications. Continuous glucose monitoring (CGM) allows for frequent, automated sensor glucose readings from interstitial fluid in the subcutaneous tissue space. CGM may be employed to improve glycemic control and reduce glycemic excursion—potentially decreasing both hypoglycemia and hyperglycemia. Additionally, CGM may be used in combination with insulin pumps via sensor augmented pump therapy. Real-time CGM devices may provide real-time auditory alerts for glucose excursions above or below customized thresholds but may not predict impending hypoglycemic events.

### SUMMARY

An embodiment of a method for predicting a hypoglycemic event or a hyperglycemic event comprises (a) collecting one or more data streams, wherein at least one of the one or more data streams comprises a glucose (GL) data stream corresponding to a user, (b) determining one or more features based on the one or more collected data streams, wherein at least one of the one or more features is distinct from each of the one or more data streams, (c) generating a prediction of whether or not a hypoglycemic event or a hyperglycemic event will occur by a prediction model based on the one or more features determined at (b), and (d) issuing an alert to the user in response to the prediction generated at (c) comprising a prediction that a hypoglycemic event or a hyperglycemic event will occur. In some embodiments, the one or more features comprises one or more short-term features of the GL data stream corresponding to a first time window preceding a current GL measurement of the user and having a first length, medium-term features of the GL data stream corresponding to a second time window preceding the current GL measurement of the user having a second length that is longer than the first length, and long-term features of the GL data stream corresponding to a third time window preceding the current GL measurement of the user having a third length that is greater than the second length. In some embodiments, the one or more data streams comprise at least one of a food intake data stream, a data stream comprising data pertaining to a current activity of the user, an insulin intake data stream, an electrocardiography (ECG) data stream, a photoplethysmography data stream, and a data stream comprising data inputted by the user. In certain embodiments, the short-term features comprise a difference between the current GL measurement and a prior GL measurement of the user observed within the first time window, and the medium-term features comprise a standard deviation of a plurality of GL measurements of the user observed within the second time window, a maximum decrease in adjacent GL measurements of the user within the second time window, and a sum of decreases in adjacent GL measurements of the user in the second time window. In certain embodiments, the one or more features comprises demographic information of the user. In some embodiments, the one or more features comprises an amount of insulin on board the user or an amount of carbohydrates on board the user. In some embodiments, the method further comprises (e) selecting a subset of the one or more features determined at (b), wherein (c) comprises generating the prediction of whether or not the hypoglycemic event or the hyperglycemic event will occur by the prediction model based on the subset selected at (e). In certain embodiments, (c) comprises generating the prediction of whether or not the hypoglycemic event or the hyperglycemic event will occur at any point in time within a predefined prediction time window.

An embodiment of a method for predicting a hypoglycemic event or a hyperglycemic event comprises (a) collecting one or more data streams, wherein at least one of the one or more data streams comprises a glucose (GL) data stream corresponding to a user, (b) determining one or more features based on the one or more collected data streams, (c) generating a prediction of whether or not a hypoglycemic event or a hyperglycemic event will occur at any point in time within a predefined prediction time window or at a specific point in time by a prediction model based on the one or more features determined at (b), and (d) issuing an alert to the user in response to the prediction generated at (c) comprising a prediction that a hypoglycemic event or a hyperglycemic event will occur at any point in time within the prediction time window or at a specific point in time. In some embodiments, the prediction time window extends between thirty minutes and sixty minutes following a current GL measurement of the user. In some embodiments, the method further comprises (e) selecting a subset of the one or more features determined at (b), wherein (c) comprises generating the prediction of whether or not the hypoglycemic event or the hyperglycemic event will occur by the prediction model based on the subset selected at (e). In some embodiments, the method further comprises (f) training the prediction model with the one or more features, wherein the one or more features comprise historical data. In certain embodiments, the method further comprises (e) selecting a nocturnal prediction model from a plurality of prediction models to generate the prediction at (c) based on an hour of the day at which a current GL measurement of the user is taken. In certain embodiments, the one or more features comprises one or more short-term features of the GL data stream corresponding to a first time window preceding a current GL measurement of the user and having a first length, medium-term features of the GL data stream corresponding to a second time window preceding the current GL measurement of the user having a second length that is longer than the first length, and long-term features of the GL data stream corresponding to a third time window preceding the current GL measurement of the user having a third length that is greater than the second length. In some embodiments, the short-term features comprise a difference between the current GL measurement and a prior GL measurement of the user observed within the first time window, and the medium-term features comprise a standard deviation of a plurality of GL measurements of the user observed within the second time window, a maximum decrease in adjacent GL measurements of the user within the second time window, and a sum of decreases in adjacent GL measurements of the user in the second time window.

An embodiment of a system for predicting a hypoglycemic event or a hyperglycemic event comprises a processor, a non-transitory memory, and one or more applications stored in the non-transitory memory that, when executed by the processor collect one or more data streams, wherein at least one of the one or more data streams comprises a glucose (GL) data stream corresponding to a user, determine one or more features based on the one or more collected data streams, wherein at least one of the one or more features is distinct from each of the one or more data streams, generate a prediction of whether or not a hypoglycemic event or a hyperglycemic event will occur by a prediction model based on the one or more determined features, and issue an alert to the user in response to the generated prediction comprising a prediction that a hypoglycemic event or a hyperglycemic event will occur. In some embodiments, the one or more features comprises one or more short-term features of the GL data stream corresponding to a first time window preceding a current GL measurement of the user and having a first length, medium-term features of the GL data stream corresponding to a second time window preceding the current GL measurement of the user having a second length that is longer than the first length, and long-term features of the GL data stream corresponding to a third time window preceding the current GL measurement of the user having a third length that is greater than the second length. In some embodiments, the one or more data streams comprise at least one of a food intake data stream, a data stream comprising data pertaining to a current activity of the user, an insulin intake data stream, an electrocardiography (ECG) data stream, a photoplethysmography data stream, and a data stream comprising data inputted by the user. In certain embodiments, the one or more applications stored in the non-transitory memory that, when executed by the processor, select a subset of the one or more features, generate a prediction of whether or not the hypoglycemic event or the hyperglycemic event will occur by the prediction model based on the selected subset. In some embodiments, the one or more applications stored in the non-transitory memory that, when executed by the processor, generate a prediction of whether or not a hypoglycemic event or a hyperglycemic event will occur at any point in time within a predefined prediction time window by a prediction model based on the one or more features.

Embodiments described herein comprise a combination of features and characteristics intended to address various shortcomings associated with certain prior devices, systems, and methods. The foregoing has outlined rather broadly the features and technical characteristics of the disclosed embodiments in order that the detailed description that follows may be better understood. The various characteristics and features described above, as well as others, will be readily apparent to those skilled in the art upon reading the following detailed description, and by referring to the accompanying drawings. It should be appreciated that the conception and the specific embodiments disclosed may be readily utilized as a basis for modifying or designing other structures for carrying out the same purposes as the disclosed embodiments. It should also be realized that such equivalent constructions do not depart from the spirit and scope of the principles disclosed herein.

### BRIEF DESCRIPTION OF THE DRAWINGS

For a detailed description of various exemplary embodiments, reference will now be made to the accompanying drawings in which:

FIG. 1 is a flowchart of a method for predicting a hypoglycemic or a hyperglycemic event according to some embodiments,

FIG. 2 is a graph of an exemplary glucose data stream including a rebound low and a near rebound low according to some embodiments,

FIG. 3 is a graph of an exemplary glucose data stream including a rebound high and a near rebound high according to some embodiments

FIG. 4 is a is a graph of exemplary glucose data according to some embodiments;

FIG. 5 is a graph illustrating predicted insulin on board over time according to some embodiments;

FIG. 6 is a is a schematic representation of a prediction model according to some embodiments

FIG. 7 is a is a graph of an error rate of a prediction model according to some embodiments;

FIG. 8 is a is a schematic view of a system for predicting a hypoglycemic or a hyperglycemic event according to some embodiments; and

FIG. 9 is a block diagram of an embodiment of a computer system according to some embodiments.

### DETAILED DESCRIPTION

The following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection of the two devices, or through an indirect connection that is established via other devices, components, nodes, and connections. As used herein, the terms “approximately,” “about,” “substantially,” and the like mean within 10% (i.e., plus or minus 10%) of the recited value. Thus, for example, a recited angle of “about 80 degrees” refers to an angle ranging from 72 degrees to 88 degrees.

As described above, CGM, which may be used in combination with insulin pumps via sensor augmented pump therapy, may be employed to improve glycemic control and reduce glycemic excursion. Particularly, CGM devices may take GL measurements or readings at a fixed frequency (e.g., every five minutes) and alert a user of the CGM device should the user's GL drop below a threshold value. However, CGM or other devices do not provide future hypoglycemic risk prediction based on current GL status and other contextual information.

Additionally, some studies have been undertaken to address the problem of prediction of hypoglycemic event using statistical modeling. These studies can be broadly divided into (i) classification-based approaches for probabilistic prediction of a future hypoglycemic event, and (ii) regression-based approaches for future GL value prediction. The term “hypoglycemic event” as used herein corresponds to a GL measurement less than 70 milligrams per deciliter (mg/dl). The term “hyperglycemic event” as used herein corresponds to a GL measurement greater than 200 mg/dl. Some studies have leveraged machine learning (ML) methodologies for hypoglycemia risk prediction. However, these studies rely on collected raw data (e.g., individual GL observations) without extracting salient features and patterns from the collected raw data. The reliance on raw data as an input to the ML algorithm may however hamper the accuracy of the ML algorithm in predicting the probability of a future hypoglycemic event. Additionally, studies may only focus on predicting a hypoglycemic event occurring at a fixed point in time in the future, which may miss hypoglycemic events which occur between a current time and the fixed point in time in the future.

Accordingly, embodiments disclosed herein include GL prediction systems and associated methods which (i) provide a novel definition of a hypoglycemic event window based on observed real-life CGM patterns, (i) include a comprehensive feature-engineering process to identify salient features influencing future hypoglycemic or hyperglycemic risk, and/or (iii) include features from contextual information such as, for example, insulin, carb intake, as features to improve the performance of a prediction model configured to predict future hypoglycemic or hyperglycemic events. Embodiments disclosed herein may predict the probability of a hypoglycemic or hyperglycemic event over a predefined event window in addition to a future point in time. For example, the prediction model may provide a prediction of a hypoglycemic or hyperglycemic event occurring within the next thirty minutes, sixty minutes, etc. Thus, in addition to predicting the probability of a hypoglycemic or hyperglycemic event occurring at a particular moment in time, embodiments disclosed herein may predict a hypoglycemic or hyperglycemic event occurring over a predetermined period of time

Embodiments disclosed herein may extract one or more features from a plurality of collected data streams, wherein each extracted feature may be separate and distinct from the collected data streams. For example, embodiments disclosed herein may extract short (less than one hour), medium (one to four hours), and long term (greater than four hours in duration) behavioral patterns from a CGM signal and use these features to improve the performance of the prediction model. The prediction model for predicting future hypoglycemic or hyperglycemic events may comprise an algorithm such as a logistic regression algorithm or a ML algorithm. Embodiments disclosed herein may also include selecting an appropriate prediction model based on contextual information. For example, a separate prediction model may be employed for night-time risk prediction due to severe sequelae of nocturnal hypoglycemia. In this example, night-time GL patterns may vary from day-time patterns due to reduced influence of physical activity, food, and insulin intake, and hence a better performing prediction model may be developed or optimized separately for nocturnal hypoglycemia prediction. The combination of features outlined above and as further described herein may result in a prediction model having superior power and accuracy in predicting future hypoglycemic or hyperglycemic events.

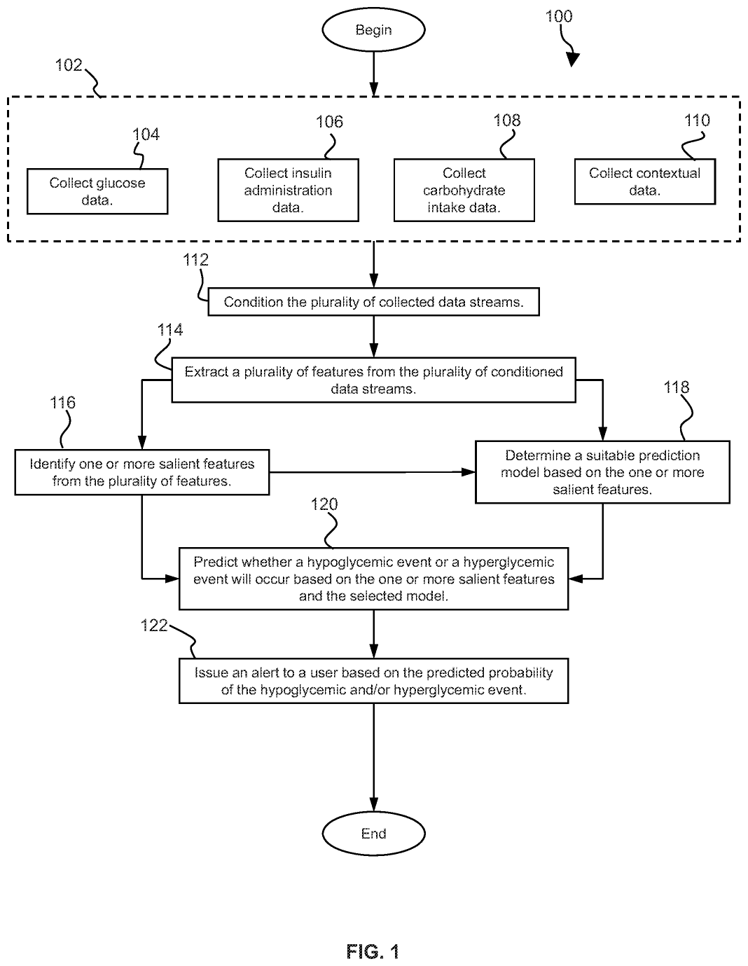

Referring initially to FIG. 1, a flowchart of a method 100 for predicting a hypoglycemic event or a hyperglycemic event is shown. Beginning at block 102, method 100 comprises collecting a plurality of data streams. The data streams collected block 102 may comprise a plurality of raw data streams. The data streams collected at block 102 may be separate and distinct from each other and may be collected from a plurality of independent sources. In an embodiment, the data collected at block 102 may be collected from a plurality of users or patients. At least some of the data collected at block 102 may be collected continuously from one or more users. As will be discussed further herein, the data collected at block 102 from the plurality of users may be used to predict whether or not a hypoglycemic event or a hyperglycemic event for one of the plurality of users. In an embodiment, a prediction of whether or not a hypoglycemic event or a hyperglycemic event based on the data collected at block 102 may be made in real-time. As used herein, the term “real-time” with reference to a prediction (a “prediction made in real-time” or a “real-time prediction”) may refer to a prediction made in at most five minutes of collecting the data. Alternatively, the data collected at block 102 may only pertain to a single user.

The data streams collected at block 102 may comprise a glucose or GL data stream 104, an insulin administration data stream 106, a carbohydrate intake data stream 108, and/or a contextual data stream 110. In other embodiments, the data collected at block 102 may vary from the data streams 104, 106, 108, and 110 shown in FIG. 1. For example, the data collected at block 102 may not include each data stream 104, 106, 108, and 110 and/or may include data streams other than data streams 104, 106, 108, and 110. Exemplary data streams collected at block 102 may include, for example, a food intake data stream, a data stream comprising data pertaining to a current activity of the user, an insulin intake data stream, an electrocardiography (ECG) data stream, a photoplethysmography data stream, and a data stream comprising data inputted by the user.

The GL data stream 104 of block 102 may be collected from one or more users at periodic intervals. For example, a glucose or GL reading or measurement may be collected from each user at approximately five minute intervals; however, the period in which GL measurements are collected from the users may vary. In an embodiment, the GL data stream 104 may be collected from one or more CGM devices coupled to one or more users. The CGM devices employed for collecting the GL data stream 104 may comprise commercially available CGM devices such as, for example, a DexCom G6® CGM device (DexCom, Inc., San Diego, Calif.); however, in other embodiments, the type of CGM device employed for collecting the GB data stream 104 may vary. In still other embodiments, the GL data stream 104 may be collected from a device other than a CGM device.

The insulin administration data stream 106 of block 102 may include the amount of insulin (e.g., in ml, for example) administered and its time of delivery. The insulin administration data stream 106 may be collected from one or more users at periodic intervals (e.g., five minute intervals, for example). Alternatively, the collection of insulin administration data may be event based and thus may be collected upon the administration of insulin to a user. In an embodiment, the insulin administration data stream 106 may be collected from one or more insulin pumps coupled to one or more users. The insulin pumps employed for collecting the insulin administration data stream 106 may comprise commercially available insulin pumps such as, for example, an Omnipod® Insulin Management System (Insulet Corp., Acton, Mass.); however, in other embodiments, the type of insulin pump employed for collecting the insulin administration data stream 106 may vary. In still other embodiments, the insulin administration data stream 106 may be collected from a device other than a CGM device.

The carbohydrate intake data stream 108 of block 102 may include the amount of carbohydrates taken by a particular user (e.g., in grams (g), for example) and the time the carbs were taken by the user. The carbohydrate intake data stream 108 may be collected from one or more users at periodic intervals (e.g., five minute intervals, for example). Alternatively, the collection of carbohydrate intake data from the one or more users may be event based and thus may be collected upon the intake of the carbohydrates by the user. For example, a user may input an amount of carbohydrates taken and the time they were taken into a portable computing device which may log the inputted carbohydrate intake, including the amount and time of the intake. In some embodiments, the computing device may comprise a smartphone and the user may input the carbohydrate intake into an application executing on the smartphone. In other embodiments, the user may input the carbohydrate intake into the insulin pump, such as a commercially available insulin pump (e.g., an Omnipod® Insulin Management System, for example).

The contextual data stream 110 of block 102 may comprise additional information specific to each user. For example, contextual data stream 110 may include demographic information pertaining to one or more users. For example, the contextual data stream 110 may include the gender, age or age group (e.g., 0-5 years, 6-10 years, etc.), the amount of time that has been passed since the user has been diagnosed with diabetes, and/or the last known Hemoglobin A1C (HbA1c) value of the user. At least some of this information may be inputted by the user via a portable computing device, such as a portable computing device used to input the carbohydrate intake data comprising the carbohydrate intake data stream 108. Contextual data stream 110. Contextual data stream 110 may additionally include information regarding the current activity of the user. For example, whether the user is currently sedentary or exercising, whether the user is currently driving, and/or whether the user is currently awake or sleeping. At least some of this information may be collected automatically by a computing device. For instance, information regarding whether the user is currently awake, exercising, sleeping, etc., may be collected by a portable computing device worn by the user having sensors configured monitor these parameters. Additionally, a user's vehicle may include a computing device configured to monitor the user's presence within the vehicle (e.g., via a short-wave radio link with a portable computing device worn by the user) and log this information.

At block 112 of method 100, the data streams collected at block 102 are conditioned. In some embodiments, block 112 may comprise integrating, aligning, and/or cleaning the data captured at block 102. For example, erroneous data, such as erroneous GL measurements made by a CGM device, for example, may be removed the data streams collected at block 102 at block 112. Additionally, a time-stamped first data stream may be aligned with a time-stamped second data stream such that the time-stamps correspond between the two data streams. In an example, CGM measurements may be collected at a periodic interval (e.g., every five minutes). Variations in the period between each period may be ignored and at block 112 and each CGM measurement may be mapped to the nearest minute to remove any minor variations between each period. Additionally, a first data stream may be integrated with a second data stream, a third data stream, etc. For example, the carbohydrate intake data stream 108 and insulin administration data stream 106 may each be temporally integrated with the glucose or GL data stream 104. Carbohydrate intake and insulin administration data may be mapped to the immediately following GL data timestamp. Outliers and/or extreme values in different data streams may be identified and approaches developed to address them.

At block 114 of method 100, a plurality of features are extracted or determined from the plurality of conditioned data streams. At least some of the features extracted at block 114 may be derived from one or more of the conditioned data streams but may be distinct from the data collected at block 102. For example, at least some of the features extracted at block 114 may comprise patterns extracted from one or more data streams collected at block 102, a manipulation of data comprising one or more data streams collected at block 102, and/or combinations of data contained in a plurality of the data streams collected at block 102. At least some of the features extracted at block 114 may comprise patterns identified within one or more of the raw data streams collected at block 102. At least some of the features extracted at block 114 may comprise observations based on one or more of the raw data streams collected at block 102, such as differences between selected data points of a particular data stream, summations of selected data points of a particular data stream, etc. The plurality of features extracted at block 114 may be selected based on their potential salience in predicting a future hypoglycemic or hyperglycemic event.

The plurality of features extracted at block 114 may comprise short-term features, medium-term features, and long-term features. Short-term features may correspond to a first time window preceding a real-time or current GL measurement, medium-term features may correspond to a second time window preceding a current GL measurement that is greater in length than the first time window, and long-term features may correspond to a third time window preceding a current GL measurement that is greater in length than the second time window. Short-term features may comprise features capturing GL variability within immediate temporal proximity of a current or real-time GL measurement or observation. In an embodiment, short-term features may comprise features capturing GL variability within less than one hour of a current GL measurement; however, in other embodiments, the temporal proximity of short-term features may vary. Short-term, medium-term, and long-term features may be determined from one or more data streams collected at block 102, including the GL data stream 104.

Short-term features may include changes in GL of one or more users within one or more predefined time windows preceding the current GL measurement of the user captured in the GL data stream 104 collected at block 102. For example, short-term features may include individual GL measurements, a difference between a current GL measurement and a GL measurement observed ten minutes prior, a difference between a current GL measurement and a GL measurement observed twenty minutes prior, a difference between a current GL measurement and a GL measurement observed thirty minutes prior, and/or a rate of change (i.e., a slope) in temporally ordered GL measurements in the hour preceding a current GL measurement. For example, and not intending to be bound by any theory, a slope Y between two temporally ordered GL measurements X₁, X₂ may be computed in accordance with the following equation:

$$Y = \frac{( {X}_{2} - {X}_{1} )}{{X}_{1}} \qquad ( 1 )$$

Medium-term features may comprise features capturing GL variability in a medium range or proximity to a current GL measurement. In an embodiment, medium-term features may comprise features capturing GL variability from one hour to four hours preceding a current GL measurement of the user; however, in other embodiments, the temporal proximity of medium-term features may vary. Medium-term features may include, for example, a standard deviation of GL measurements observed in the two hours preceding a current GL measurement, a standard deviation of GL measurements observed in the four hours preceding a current GL measurement, and/or a rate of change (i.e., a slope) in temporally ordered GL measurements in the two hour preceding a current GL measurement.

Long-term features may comprise features capturing the long-term GL profile of a particular user. In an embodiment, long-term features may comprise features capturing GL variability occurring more than hours preceding a current GL measurement; however, in other embodiments, the temporal proximity of long-term features may vary. The long-term features may encapsulate the long-term ability of one or more users to manage GL. Long-term features may include, for example, a number of rebound highs observed for a user, a number of rebound lowers observed for a user, a number of near rebound highs observed for a user, a number of near rebound lows observed for a user, a standard deviation of all GL measurements for a particular user, a percentage of GL measurements of a particular user that are less than 70 mg/dl, and/or a percentage of GL measurements of a particular user that are above 200 mg/dl. Rebound highs and rebound lows may indicate the tendency of a user to over compensate to a hypoglycemic event by taking a larger than required carbohydrate intake or a tendency to miss insulin dosage for food.

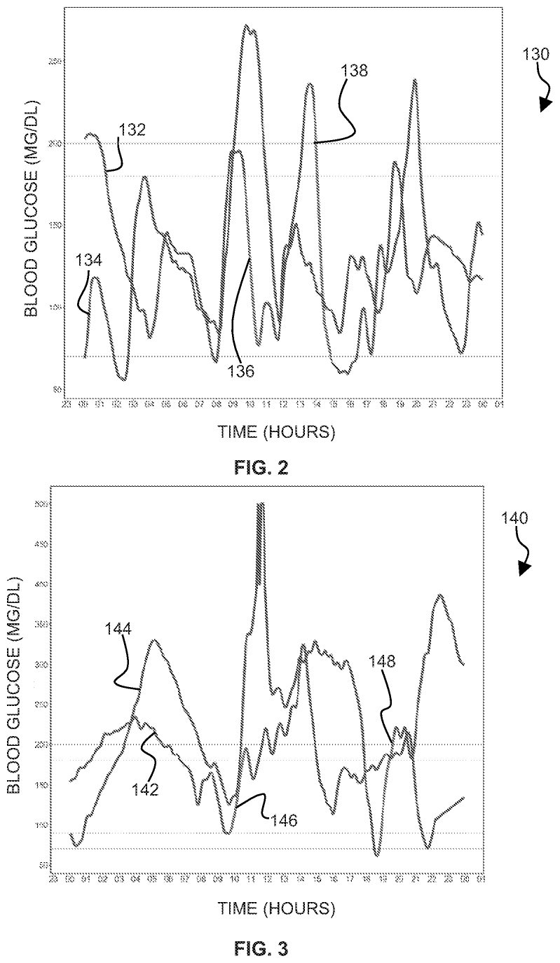

Rebound lows may comprise events in which GL is above 200 mg/dl and falls below 70 mg/dl within an hour from the GL measurement that is above 200 mg/dl. Near rebound lows may comprise events in which GL is above 180 mg/dl and falls below 90 mg/dl within an hour from the GL measurement that is above 180 mg/dl. For example, referring briefly to FIG. 2, a graph 130 is shown of an exemplary pair of GL data streams 132, 134 (GL in mg/dl indicated on the Y-axis of graph 130) of a pair of users over a particular period of time (time in hours indicated along the X-axis of graph 130). The GL data streams 132, 134 may be collected, for example, by a CGM device as described above with respect to GL data stream 104 of block 102. In this example, a first GL data stream 132 of a first user includes a near rebound low 136 between approximately 10:00 and 11:00 hours while a second GL data stream 134 of a second user includes a rebound low 138 between 14:00 and 15:00 hours.

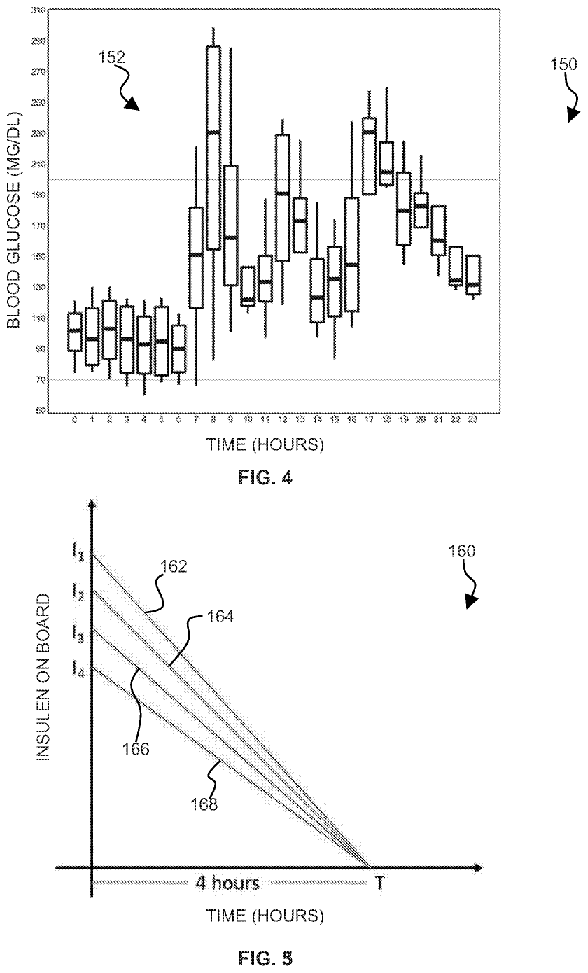

Referring again to FIG. 1, rebound highs may comprise events in which GL is less than 70 mg/dl and rises above 200 mg/dl within an hour from the GL measurement that is less than 70 mg/dl. Near rebound highs may comprise events in which GL is less than 90 mg/dl and rises above 180 mg/dl within an hour from the GL measurement that is less than 90 mg/dl. For example, referring briefly to FIG. 3, a graph 140 is shown of an exemplary pair of GL data streams 142, 144 (GL in mg/dl indicated on the Y-axis of graph 140) of a pair of users over a particular period of time (time in hours indicated along the X-axis of graph 140). The GL data streams 142, 144 may be collected, for example, by a CGM device as described above with respect to GL data stream 104 of block 102. In this example, a first GL data stream 142 of a first user includes a near rebound high 146 between approximately 10:00 and 11:00 hours while a second GL data stream 144 of a second user includes a rebound high 148 between 14:00 and 15:00 hours.

Referring again to FIG. 1, the features extracted at block 114 may include features in addition to the short-term, medium-term, and long-term features described above. Particularly, in an embodiment, the extracted features may include demographic features, such as one or more of the features captured in the contextual data stream 110 at block 102 (e.g., gender, age or age group, years since diabetes diagnosis, and/or last known HbA1c value, etc.). In an embodiment, the extracted features may include additional features which contextualize one or more of the data streams collected at block 102. For example, the extracted features may include contextual features such as an hour of the day in which a particular measurement (e.g., a GL data for a particular user) was made, a day of the week in which a particular measurement was made, an amount of insulin “on board” a particular user at a given time, and an amount of carbohydrates “on board” a particular user at a given time.

GL patterns for a particular user may vary significantly throughout a given day, and thus at least some of the contextual features (e.g., hour of the day of a given measurement, day of the week of a given measurement, etc.) may capture these daily and/or weekly patterns (at least some of these patterns may be repeatable) such that they may be taken account of when predicting a future hypoglycemic and/or a hyperglycemic event. For example, referring briefly to FIG. 4, a box plot 150 is shown of exemplary GL data 152 (GL in mg/dl indicated on the Y-axis of graph 150) of a particular user over a particular period of time (time in hours indicated along the X-axis of graph 150). GL data 152 indicates an exemplary daily pattern of GL measurements. Particularly, the GL data 152 illustrated in box plot 150 includes hourly median (represented by a black line in FIG. 4 extending through each hourly box comprising GL data 152) and quarterlies (represented by the upper and lower bounds of each hourly box comprising GL data 152) of GL measurements performed during each given hour of the day.

Referring again to FIG. 1, insulin on board may comprise an amount of insulin (e.g., in mg/dl) remaining in a body of a user at a given point in time. Insulin on board for a given user may be determined from the insulin administration data stream 106 pertaining to the user and collected at block 102. Insulin on board may be a function of the amount of insulin administered at a given time and the amount of time that has passed since the administration of the insulin. For example, referring briefly to FIG. 5, a graph 160 is shown of a plurality of modeled or predicted insulin on board amounts 162, 164, 166, and 168 (unitless insulin on board mount indicated on the Y-axis of graph 160) over time (time in hours indicated along the X-axis of graph 160). In this example, different amounts of insulin I₁ (corresponding to insulin on board 162), I₂ (corresponding to insulin on board 164), I₃ (corresponding to insulin on board 166), and I₄ (corresponding to insulin on board 166) are administered to different users at a first point in time (T=0). In this example, insulin amount I₁ exceeds insulin amounts I₂, I₃, and I₄; insulin amount I₂ exceeds insulin amounts I₃, and I₄, and insulin amount I₃ exceeds insulin amount I₄. The amount of predicted insulin on board 162, 164, 166, and 168 each user decreases linearly following the first point in time until the amount of insulin on board 162, 164, 166, and 168 is approximately zero at a second point in time (T=four hours in this example). In this example, the predicted insulin on board amounts 162, 164, 166, and 168 are based on a linear model where the reduction in insulin on board over time decreases linearly with a slope that is proportional to the amount of insulin initially administered. Thus, the slope of the first predicted insulin on board 162 is greater than the slopes of predicted insulin on boards 164, 166, 168, and so on and so forth. In this exemplary model, it is assumed the effects of insulin administration wear off after four hours, resulting in the predicted insulin on board amounts 162, 164, 166, and 168 equaling zero at T=four hours.

Referring again to FIG. 1, an amount of carbohydrates “on board” a particular user at a given time may also be predicted using models, including linear models. In an example, a feature comprising carbohydrates on board a particular user may be extracted at block 114 using a linear model which assumes carbohydrates are absorbed at an approximate rate of approximately 0.5 grams per minute (g/min) after an initial lag of approximately 15 minutes; however, varying models may be employed to predict remaining carbohydrates on board following an initial intake of carbohydrates by a user.

The features extracted at block 114 may also include snowball effect features for one or more users. Snowball effect features may capture the accruing effects of changes in GL measurements over time. For example, snowball effect features extracted at block 114 may include a sum of all increments in adjacent GL measurements for a particular user in the two hours preceding a current GL measurement, a sum of all decrements in adjacent GL measurements for a particular user in the two hours preceding a current GL measurement, a maximum increase in adjacent GL measurements for a particular user in the two hours preceding a current GL measurement, and a maximum decrease in adjacent GL measurements for a particular user in the two hours preceding a current GL measurement; however, in other embodiments, the snowball effect features extracted at block 114 may vary.

The features extracted at block 114 may further include interaction and nonlinear features for one or more users. For hypoglycemia, GL reductions at a higher base GL may not be as salient as GL reductions at a lower base GL. For example, a reduction in GL of 10 mg/dl is more serious when GL is initially at 70 mg/dl than when GL is initially at 200 mg/dl. Given this phenomenon, features extracted at block 114 may include an interaction between a current GL measurement for a particular user and an overall standard deviation of GL measurements for the user, an interaction between a current GL measurement for a particular user and a difference in GL measurements for the user in the ten minutes preceding the current GL measurement, and a square of the current GL measurement for a particular user; however, in other embodiments, interaction and nonlinear features extracted at block 114 may vary. Additionally, the snowball effect features and interaction and nonlinear features described above may be based on the CGM data stream 104 collected at block 102 for one or more users. In an embodiment, a total of approximately 25 to 30 features may be extracted at block 114; however, in other embodiments, the number of features extracted at block 114 may vary.

Method 100 proceeds at block 116 which comprises identifying one or more salient features from the plurality of features extracted at block 114. In an embodiment, block 116 comprises developing or optimizing one or more prediction models for predicting whether or not a hypoglycemic event or a hyperglycemic event will occur. The one or more prediction models optimized at block 116 may comprise a regression model, such as a linear regression model, and/or a classification model, such as a logistic regression (LR) or random forest model. Block 116 may comprise applying at least some of the features extracted at block 114 to one or more un-optimized or base prediction models as train and/or test data to produce the one or more optimized prediction models and to identify the one or more salient features of the plurality of features extracted at block 114. Data collected from different users may be partitioned in different ways to develop or train and to test or evaluate the one or more models.

In an embodiment, the one or more prediction models optimized at block 116 may comprise one or more Random Forest (RF) ML models. RF models may comprise classification models that include an ensemble classifier comprising multiple randomized decision trees. RF models may extend trees by generating multiple trees, with each tree developed on a bootstrapped sample of the data (e.g., the plurality of features extracted at block 114). The number of features used in a split at a node may be randomly restricted to a subset of the features identified at block 114. A final classification prediction may be made based on a classification predicted by a majority of the developed trees of the RF model.

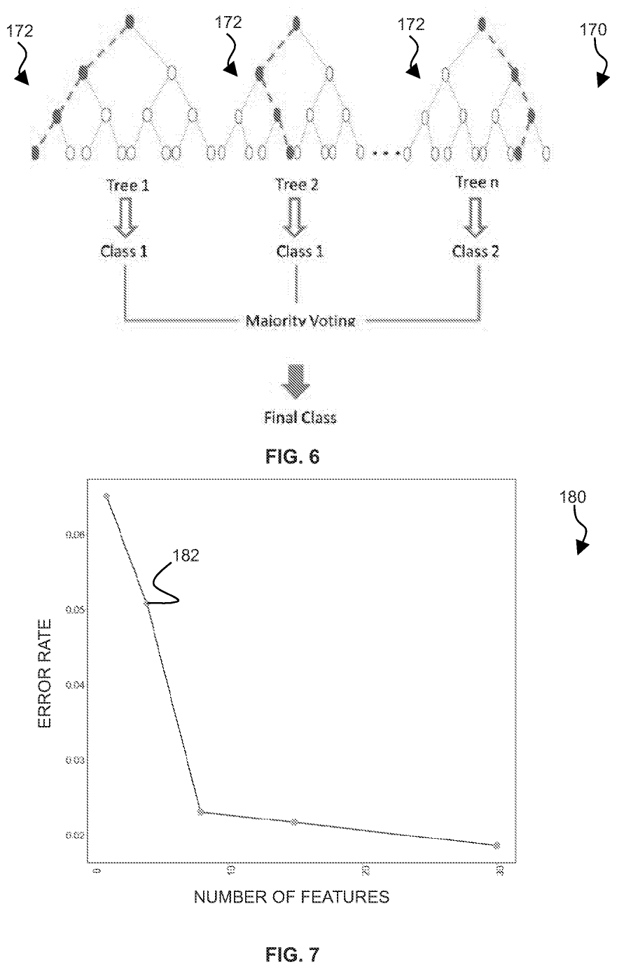

Referring briefly to FIG. 6, a schematic representation of an exemplary RF model 170 is shown. RF model 170 comprises a plurality of trees 172, each tree receiving as an input a sample of the plurality of features extracted at block 114. Each tree 172 of RF model 170 may output a classification comprising a prediction based on the input supplied to the particular tree 172. In some embodiments, the classifications outputted by each tree 172 may comprise a prediction that a hypoglycemic event will (“Class 2”) or will not occur (“Class 1”) within a predefined period of time following a current time (e.g., a time at which the prediction is made). Alternatively, the classifications outputted by each tree 172 may comprise a prediction that a hyperglycemic event will (“Class 2”) or will not occur (“Class 1”) within a predefined period of time following a current time (e.g., a time at which the prediction is made). A final prediction or classification may be outputted by the RF model 170 through a majority vote among the trees 172 of the RF model 170.

As described above, the final classification outputted by the RF model 170 may comprise a prediction of whether a hypoglycemic or a hyperglycemic event will or will not occur within a predefined period time. In other words, the RF model 170 may predict whether a hypoglycemic or a hyperglycemic event will or will not occur at any point in time within a predefined time window, in addition to predicting whether a hypoglycemic or a hyperglycemic event will or will not occur at a particular future point in time. RF model 170 may predict the occurrence of a hypoglycemic or a hyperglycemic event throughout an entire time window (e.g., a thirty minute window, a sixty minute window, a four hour window, etc.) in addition to predicting at a particular point in time. By covering an entire time window, RF model 170 may capture instances where GL of a particular user may fall below 70 mg/dl or rise above 200 mg/dl at one point within the time window (e.g., at 15 minutes within a thirty minute time window, for example) but may rebound above 70 mg/dl or below 200 mg/dl by the conclusion of the time window (e.g., at thirty minutes in this example). A prediction model only capable of predicting the occurrence of a hypoglycemic or a hyperglycemic event at the conclusion of the time window may thus miss an occurrence of a hypoglycemic or a hyperglycemic event that occurs within but prior to the conclusion of the time window.

Referring again to FIG. 1, in some embodiments at least some of the features extracted at block 114 may comprise historical data pertaining to one or more users and configured to train and test the one or more prediction models (e.g., one or more RF models) and thereby to identify the one or more salient features. For example, one or more salient features may be identified at block 116 using one or more RF models and a Variable Importance Factor (VIF) plot that is based on node purity and out of bag (OOB) error estimates. Particularly, misclassification rates associated with different numbers of the features extracted at block 114 may be determined using OOB estimates based on the bootstrapped samples supplied to the RF model.

Referring briefly to FIG. 7, a graph 180 illustrating an exemplary error or misclassification rate 182 for a RF model as a function of the number of features (e.g., features extracted at block 114) provided to the RF model. As indicated by graph 180, error rate 182 may decrease as additional features are provided to the RF model. Particularly, the error rate 182 may increase substantially when fewer than nine of the plurality of features (e.g., nine of the twenty six exemplary features described above) extracted at block 114 are used. It may be understood that the error rate of other prediction models using other features may vary from the error rate 182 shown in FIG. 7.

Referring again to FIG. 1, in view of the increase in error rate 182 in FIG. 7 when fewer than nine features are used, in an embodiment, a trained RF model may be optimized at block 116 which uses the nine most salient features of the plurality of features extracted at block 114, where the nine most salient features are identified as the nine features having the highest node purity. Particularly, A VIF plot may capture average improvement in class purity for splits involving a feature across all of the trees of the RF model. A VIF plot may be used to order features (e.g., the plurality of features extracted at block 114) based on their misclassification impact, and features with marginal impact may be excluded to streamline the trained RF model and thereby increase the speed in which the RF model may predict a probability of a hypoglycemic or a hyperglycemic event. To state in other words, the most salient features identified at block 116 may comprise the features having the greatest impact in minimizing an error or misclassification rate of a prediction model, such as an RF model.

In an embodiment, the salient features identified at block 116 may comprise a current hour of the day; a current day of the week; a current GL measurement of the user; a standard deviation of GL measurements of the user observed in the two hours preceding a current GL measurement; a standard deviation of GL measurements of the user observed in the four hours preceding a current GL measurement; a difference between a current GL measurement of the user and a GL measurement taken ten minutes prior to the current GL measurement; a sum of increases in adjacent GL measurements of the user within two hours of a current GL measurement of the user; a maximum positive increase in adjacent GL measurements of the user within two hours of a current GL measurement of the user; a sum of decreases in adjacent GL measurements of the user within two hours of a current GL measurement of the use; and a maximum decrease in adjacent GL measurements of the user within two hours of a current GL measurement of the user. In other embodiments, the salient features identified at block 116 may vary from the features listed above.

In an embodiment, developing or optimizing one or more prediction models (e.g., a RF model) may comprise identifying the salient features using an un-optimized prediction model and then applying the identified salient features to the prediction model. Although the development of one or more RF models is described above, in other embodiments, prediction models other than RF models may be employed to predict a probability of a hypoglycemic or a hyperglycemic event. For example, an un-optimized logistic regression model may be used to identify one or more salient features at block 116. The identified salient features may then be applied to a now optimized logistic regression model to predict a probability of a hypoglycemic or a hyperglycemic event. In an embodiment, Least Absolute Shrinkage and Selection Operator (LASSO) may be employed to identify one or more salient features from the plurality of features extracted at block 114. Generally, LASSO employed through a LR model may identify one or more salient features by penalizing each additional nonzero parameter coefficient in the LR model by a tuning parameter. In still other embodiments, other prediction models may be used such as, for example, Quantile Regression Forests (QRF) models.

Method 100 continues at block 118 which comprises determining a suitable prediction model based on the salient features identified at block 116. In an embodiment, block 118 comprises selecting one of the prediction models optimized at block 116 based on the salient features identified at block 116. For example, block 118 may comprise selecting between a standard prediction model (e.g., a RF model) optimized at block 116 for the whole day, a diurnal prediction model optimized for only day time, and a nocturnal prediction model optimized for night time (e.g., between 23:00 hours and 6:00 hours). Also, the optimized model, and features used in the optimized model may be refined based on operational considerations on collecting, storing, and processing the features.

The standard, diurnal, and nocturnal prediction models may each rely on different sets of salient features to optimize their predictive powers (accuracy, sensitivity, and/or specificity) for the whole day in the case of the standard prediction model, during the day only for the diurnal prediction model, and during at night only for the nocturnal prediction model. Generally, insulin boluses, carbohydrate intake, and physical activity may have a lesser effect at night as the user sleeps, and thus a prediction model optimized for night time only may have greater predictive power than a prediction model that is not optimized for night time only. Moreover, the consequences of nocturnal hypoglycemia may be relatively more severe than the consequences of diurnal hypoglycemia given that user suffering from nocturnal hypoglycemia may be asleep and thus unable to immediately take corrective action to address the hypoglycemic event. Thus, it may be advantageous to employ a prediction model optimized specifically for night time to ensure a prediction model is employed that is as accurate, sensitive (the proportion of true positives that are correctly identified), and specific (the proportion of actual negatives that are correctly identified) as possible in predicting a probability of a nocturnal hypoglycemic event whereby a user may be roused (e.g., via an alert issued in response to a prediction of a future hypoglycemic event) from sleep to address the predicted hypoglycemic event.

The selection of one of the prediction models optimized at block 16 may be based on one or more of the features extracted at block 114. In an embodiment, the selection may be based on the contextual feature of the current hour of the day. For example, if the current hour of the day is between 23:00 hours and 6:00 hours then a nocturnal prediction model, such as a nocturnal RF model, optimized for the night time only may be selected at block 118. However, if the current hour of the day is not between 23:00 hours and 6:00 hours, then either a prediction model optimized for the entire day or a prediction model optimized for day time only may be selected at block 118. In other embodiments, contextual features and/or features other than contextual features may be used to select between the one or more prediction models optimized at block 116.

Method 100 continues at block 120 which comprises predicting whether or not a hypoglycemic event or a hyperglycemic event will occur based on the one or more salient features identified at block 116 and the prediction model selected at block 118. In an embodiment, block 120 comprises predicting a probability of a hypoglycemic/hyperglycemic event using an optimized prediction model (e.g., a whole day prediction model, a night time prediction model, or a day time prediction model) selected at block 118, the selected optimized prediction model having a set of corresponding salient features which are applied thereto to generate the prediction of the probability the hypoglycemic/hyperglycemic event. Unlike the features used to train or optimize the selected prediction model (e.g., an optimized RF model), which may be based on historical data pertaining to a plurality users, the salient features employed by the optimized prediction model in generating the prediction may be specific to the particular user about which the prediction is being made. Additionally, the salient features employed by the optimized prediction model in generating the prediction may be based on current or real-time data collected at block 102, such as a current GL measurement which may be provided by, for example, a CGM device worn by the user. Thus, in generating a prediction of a probability that a particular user will undergo a hypoglycemic/hyperglycemic event, the salient features relied upon by the optimized prediction model as an input may be tailored or otherwise specific to the particular user.

In an embodiment, the prediction provided at block 120 may be a prediction of a hypoglycemic event or a hyperglycemic event occurring within a predefined prediction time window. For example, a prediction may be provided at block 120 that a hypoglycemic event or a hyperglycemic event will or will not occur within thirty minutes, sixty minutes, two hours, etc., from the current time. Thus, if the optimized prediction model predicts that a hypoglycemic event or a hyperglycemic event will occur at some point in time within the prediction time window, then the optimized prediction model will issue a positive prediction of the occurrence of a hypoglycemic event or a hyperglycemic event. Conversely, if the optimized prediction model predicts that at no point in time within the prediction time window a hypoglycemic event or a hyperglycemic event will occur, then the optimized prediction model will issue a negative prediction of the occurrence of a hypoglycemic event or a hyperglycemic event.

The threshold probability of a hypoglycemic event or a hyperglycemic event required to issue a positive prediction may be configured to optimize prediction performance results. Additionally, the threshold probability may be a function of one or more features extracted at block 114. For example, the threshold probability required to issue a positive prediction may be reduced if the current time is at night (e.g., between 23:00 hours and 6:00 hours) relative to the threshold probability required to issue a positive prediction during daytime hours. The threshold probability may also be based on the type of activity in which the particular user is engaged. For example, the threshold probability may be decreased if the user is currently engaged in a hazardous activity, such as driving, given that the consequences of a hypoglycemic/hyperglycemic event occurring during the performance of a hazardous activity may be elevated.

Method 100 proceeds at block 122 which comprises delivering an alert to a user based on the probability of a hypoglycemic or a hyperglycemic event predicted at block 120. In an embodiment, block 122 comprises issuing an alert to the user in response to an optimized prediction model (e.g., an optimized RF model) issuing a positive prediction at block 120. For example, an alert may be communicated to and issued from a portable computing device in proximity to the user, such as a smartphone or other device worn by the user. The alert may also be issued through computing devices connected to the portable computing device, such as through the speakers of a vehicle connected to the portable device through short-wave radio signals. Alerts may also be delivered through haptic devices and smart devices such as smart lights.

The alert issued at block 122 may be audible, visual, audiovisual or haptic and may be configured to obtain the user's attention whereby the user may perform a corrective action to address the predicted hypoglycemic/hyperglycemic event such as through the administration of insulin or carbohydrate intake. In an embodiment, the type of alert issued may be based on one or more features extracted at block 114. For example, the alert may be more pronounced (e.g., issued at a louder volume, a higher brightness, etc.) if the current time is at night (e.g., between 23:00 hours and 6:00 hours) and the user may potentially be at sleep.

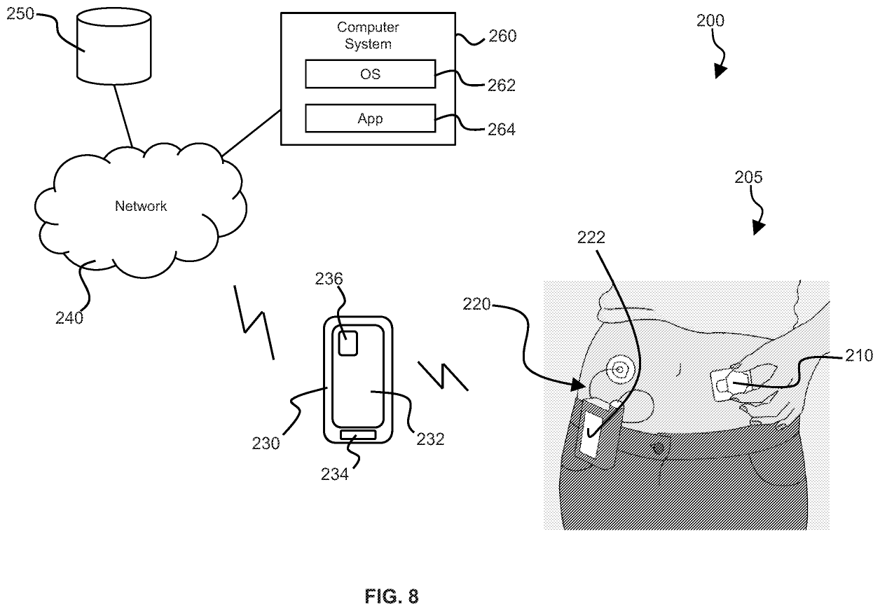

Referring to FIG. 8, an embodiment of a system 200 for predicting a hypoglycemic event or a hyperglycemic event is shown. System 200 may generally include a CGM device 210, an insulin pump 220, a portable computing device or system 230, a data store 250, and a computer system 260. CGM device 210 may be generally configured to perform GL measurements of a user or patient 205 at a fixed frequency (e.g., every minute, every five minutes, every ten minutes, etc.). CGM device 210 may comprise a commercially available CGM device such as, for example, a DexCom G6® CGM device (DexCom, Inc., San Diego, Calif.); however, in other embodiments, the type of CGM device 210 used in system 200 may vary.

Insulin pump 220 of system 200 may be generally configured to selectably administer a known or predefined amount of insulin to the user 205. Insulin pump 220 may administer insulin to user 205 automatically or in response to an input provided to a user interface 222 of the insulin pump 220 by the user 205. Additionally, insulin pump 220 may receive inputs from the user 205 indicating carbohydrates taken (e.g., an amount of carbohydrates and a time at which the amount was taken) by the user 205 whereby the amount of carbohydrates and the times at which the amounts of carbohydrates were taken may be tracked by the insulin pump 220. In an embodiment, insulin pump 220 may comprise a commercially available insulin pump such as, for example, the Omnipod® Insulin Management System (Insulet Corp., Acton, Mass.); however, in other embodiments, the type of insulin pump 220 used in system 200 may vary.

The portable computing device 230 of system 220 may generally include a user interface 232 (e.g., a touchscreen, etc.) and a speaker 234. Portable computing device 230 may comprise an application 236 which executes upon an operating system of the computing device 230. User 205 may interact with the application 236 via the user interface 232 of portable computing device 230. The application 236 may be configured to interface between CGM device 210, insulin pump 220, and the computer system 260. Particularly, each of the CGM device 210 and insulin pump 220 may comprise a wireless transceiver whereby the CGM device 210 and insulin pump 220 may each communicate wirelessly with the portable computing device 230 via a wireless transceiver of the computing device 230. For example, CGM device 210 and insulin pump 220 may each be connectable to the portable computing device 230 via one or more Bluetooth® or other wireless short-wave radio links formed between the computing device 230 and the CGM device 210 and insulin pump 220.

Application 236 may interact with the CGM device 210 and/or insulin pump 220 via the wireless links described above. Particularly, application 236 may control the operation of CGM device 210 and/or insulin pump 220. For example, application 236 may control the rate at which CGM device 210 performs GL measurements on user 205 and/or may control the administration of insulin to user 205 from insulin pump 220. Application 236 may also receive and store in a memory of portable computing device 230 data transmitted to computing device 230 from CGM device 210 and/or insulin pump 220 such as, for example, GL measurements, insulin administration data, carbohydrate intake data, etc. Application 236 may also relay data transmitted to computing device 230 from CGM device 210 and/or insulin pump 220 to the computer system 260. Application 236 may also receive data inputs from the user 205 via the user interface 232 of portable computing device 230. For example, the user 205 may input biographical data (e.g., gender, age or age group, how long ago the user 205 was diagnosed with diabetes, last known HbA1c value, etc.) into the application 236. The user 205 may also input carbohydrate intake data into the application 236 rather than the insulin pump 220.

Application 236 executing on portable computing device 230 may interface with other computing devices to acquire additional data. For example, application 236 may interface with a computing device associated with a vehicle (e.g., via a wireless link) to collect data pertaining to whether or not user 205 is currently driving. Application 236 may also acquire data pertaining to an activity level of the user 205 (e.g., whether the user 205 is currently exercising, etc.) either directly from features of portable computing device 230 or from another computing device worn by the user 205 and in signal communication with portable computing device 230.

Portable computing device 230 may be communicatively coupled with computer system 260 via a network 240. The network 240 of system 200 may comprise one or more public networks, one or more private networks, or a combination thereof. While shown as communicatively coupled to the network 240, data store 250 and computer system 260 may be considered part of network 240 and are illustrated as separate from network 240 in FIG. 8 to promote discussing their roles with respect to portable device 230.

Computer system 260 may comprise a server of network 240 and may include an operating system (OS) 262 and an application 264 executing on the OS 262. Application 264 may be configured to perform at least some of the steps of method 100 shown in FIG. 1 and may comprise or be configured to invoke one or more prediction models executing on the OS 262 of computer system 260. For example, application 264 may be configured to condition one or more data streams (e.g., provided by CGM device 210, insulin pump 220, and/or application 236 of portable computing device 230), extract a plurality of features from the one or more conditioned data streams, identify one or more salient features from the plurality of extracted features, determine a suitable prediction model based on the one or more features, predict whether a hypoglycemic event or a hyperglycemic event will occur based on the one or more salient features and the selected prediction model, and/or issue an alert to a user (e.g., user 205) based on the generated prediction. To state in other words, application 264 of computer system 260 may be configured to perform each, or at least some of, blocks 112, 114, 116, 118, 120, and 122 of method 100 shown in FIG. 1. However, in other embodiments, application 236 of portable computing device 230, rather than application 264 of computer system 260 may perform each, or at least some of, blocks 112, 114, 116, 118, 120, and 122 of method 100 shown in FIG. 1

Application 264 of computer system 260 may be configured to issue an alert to a user 205 using the user interface 232 and/or speaker 234 of portable computing device 230. Application 264 may also interface with other computing devices to route an alert to the user 205. In an embodiment, data store 250 may comprise a repository of collected data pertaining to user 205 (e.g., data corresponding to data streams 104, 106, 108, and/or 110 shown in FIG. 1) as well as other users. Data store 250 may also house historical data used to train and test one or more prediction models executing on computer system 260. However, in other embodiments, data pertaining to user 205 may be stored in a memory of the portable computing device 230 rather than in a separate data store, such as data store 250.

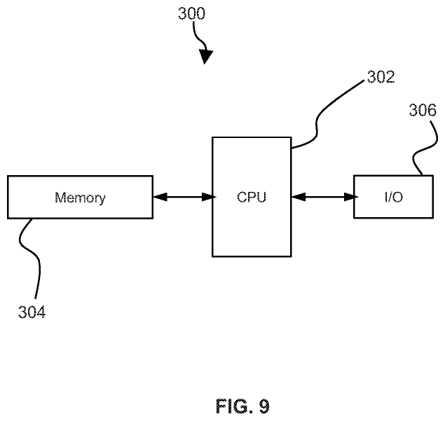

Referring to FIG. 9, an embodiment of a computer system 300 suitable for implementing one or more embodiments disclosed herein is shown. For example, components of the system 200 (e.g., portable computing device 230, computer system 260, etc.) shown in FIG. 8 may be configured in a manner similar to the computer system 300 shown in FIG. 9. The computer system 300 includes a processor 302 (which may be referred to as a central processor unit or CPU) that is in communication with one or more memory devices 304, and input/output (I/O) devices 306. The processor 302 may be implemented as one or more CPU chips. The memory devices 304 of computer system 300 may include secondary storage (e.g., one or more disk drives, etc.), a non-volatile memory device such as read only memory (ROM), and a volatile memory device such as random access memory (RAM). In some contexts, the secondary storage ROM 306, and/or RAM comprising the memory devices 304 of computer system 300 may be referred to as a non-transitory computer readable medium or a computer readable storage media. I/O devices 306 may include printers, video monitors, liquid crystal displays (LCDs), touch screen displays, keyboards, keypads, switches, dials, mice, and/or other well-known input devices.

It is understood that by programming and/or loading executable instructions onto the computer system 300, at least one of the CPU 302, the memory devices 304 are changed, transforming the computer system 300 in part into a particular machine or apparatus having the novel functionality taught by the present disclosure. Additionally, after the computer system 300 is turned on or booted, the CPU 302 may execute a computer program or application. For example, the CPU 302 may execute software or firmware stored in the memory devices 304. The software stored in the memory devices 304 and executed by CPU 302 may comprise the screen-out predictor module 250 shown in FIG. 5. During execution, an application may load instructions into the CPU 302, for example load some of the instructions of the application into a cache of the CPU 302. In some contexts, an application that is executed may be said to configure the CPU 302 to do something, e.g., to configure the CPU 302 to perform the function or functions promoted by the subject application. When the CPU 302 is configured in this way by the application, the CPU 302 becomes a specific purpose computer or a specific purpose machine.

While exemplary embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. For example, the configuration of particular data streams, the ways in which data comprising various data streams may be collected, the type and number of features relied upon for making predictions, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

What is claimed is:

1. A method for predicting a hypoglycemic event or a hyperglycemic event, comprising:
(a) collecting one or more data streams, wherein at least one of the one or more data streams comprises a glucose (GL) data stream corresponding to a user;
(b) determining one or more features based on the one or more collected data streams, wherein at least one of the one or more features is distinct from each of the one or more data streams;
(c) generating a prediction of whether or not a hypoglycemic event or a hyperglycemic event will occur by a prediction model based on the one or more features determined at (b); and
(d) issuing an alert to the user in response to the prediction generated at (c) comprising a prediction that a hypoglycemic event or a hyperglycemic event will occur.

2. The method of claim 1, wherein the one or more features comprises one or more short-term features of the GL data stream corresponding to a first time window preceding a current GL measurement of the user and having a first length, medium-term features of the GL data stream corresponding to a second time window preceding the current GL measurement of the user having a second length that is longer than the first length, and long-term features of the GL data stream corresponding to a third time window preceding the current GL measurement of the user having a third length that is greater than the second length.

3. The method of claim 1, wherein the one or more data streams comprise at least one of a food intake data stream, a data stream comprising data pertaining to a current activity of the user, an insulin intake data stream, an electrocardiography (ECG) data stream, a photoplethysmography data stream, and a data stream comprising data inputted by the user.

4. The method of claim 2, wherein:
the short-term features comprise a difference between the current GL measurement and a prior GL measurement of the user observed within the first time window; and
the medium-term features comprise a standard deviation of a plurality of GL measurements of the user observed within the second time window, a maximum decrease in adjacent GL measurements of the user within the second time window; and a sum of decreases in adjacent GL measurements of the user in the second time window.

5. The method of claim 1, wherein the one or more features comprises demographic information of the user.

6. The method of claim 1, wherein the one or more features comprises an amount of insulin on board the user or an amount of carbohydrates on board the user.

7. The method of claim 1, further comprising:
(e) selecting a subset of the one or more features determined at (b);
wherein (c) comprises generating the prediction of whether or not the hypoglycemic event or the hyperglycemic event will occur by the prediction model based on the subset selected at (e).

8. The method of claim 1, wherein (c) comprises generating the prediction of whether or not the hypoglycemic event or the hyperglycemic event will occur at any point in time within a predefined prediction time window.

9. A method for predicting a hypoglycemic event or a hyperglycemic event, comprising:
(a) collecting one or more data streams, wherein at least one of the one or more data streams comprises a glucose (GL) data stream corresponding to a user;
(b) determining one or more features based on the one or more collected data streams;
(c) generating a prediction of whether or not a hypoglycemic event or a hyperglycemic event will occur at any point in time within a predefined prediction time window or at a specific point in time by a prediction model based on the one or more features determined at (b); and
(d) issuing an alert to the user in response to the prediction generated at (c) comprising a prediction that a hypoglycemic event or a hyperglycemic event will occur at any point in time within the prediction time window or at a specific point in time.

10. The method of claim 9, wherein the prediction time window extends between thirty minutes and sixty minutes following a current GL measurement of the user.

11. The method of claim 9, further comprising:
(e) selecting a subset of the one or more features determined at (b);
wherein (c) comprises generating the prediction of whether or not the hypoglycemic event or the hyperglycemic event will occur by the prediction model based on the subset selected at (e).

12. The method of claim 11, further comprising:
(f) training the prediction model with the one or more features, wherein the one or more features comprise historical data.

13. The method of claim 9, further comprising:
(e) selecting a nocturnal prediction model from a plurality of prediction models to generate the prediction at (c) based on an hour of the day at which a current GL measurement of the user is taken.

14. The method of claim 9, wherein the one or more features comprises one or more short-term features of the GL data stream corresponding to a first time window preceding a current GL measurement of the user and having a first length, medium-term features of the GL data stream corresponding to a second time window preceding the current GL measurement of the user having a second length that is longer than the first length, and long-term features of the GL data stream corresponding to a third time window preceding the current GL measurement of the user having a third length that is greater than the second length.

15. The method of claim 14, wherein:
the short-term features comprise a difference between the current GL measurement and a prior GL measurement of the user observed within the first time window; and
the medium-term features comprise a standard deviation of a plurality of GL measurements of the user observed within the second time window, a maximum decrease in adjacent GL measurements of the user within the second time window; and a sum of decreases in adjacent GL measurements of the user in the second time window.

16. A system for predicting a hypoglycemic event or a hyperglycemic event, comprising
a processor;
a non-transitory memory; and
one or more applications stored in the non-transitory memory that, when executed by the processor:
collect one or more data streams, wherein at least one of the one or more data streams comprises a glucose (GL) data stream corresponding to a user;
determine one or more features based on the one or more collected data streams, wherein at least one of the one or more features is distinct from each of the one or more data streams;
generate a prediction of whether or not a hypoglycemic event or a hyperglycemic event will occur by a prediction model based on the one or more determined features; and
issue an alert to the user in response to the generated prediction comprising a prediction that a hypoglycemic event or a hyperglycemic event will occur.

17. The system of claim 16, wherein the one or more features comprises one or more short-term features of the GL data stream corresponding to a first time window preceding a current GL measurement of the user and having a first length, medium-term features of the GL data stream corresponding to a second time window preceding the current GL measurement of the user having a second length that is longer than the first length, and long-term features of the GL data stream corresponding to a third time window preceding the current GL measurement of the user having a third length that is greater than the second length.

18. The system of claim 16, wherein the one or more data streams comprise at least one of a food intake data stream, a data stream comprising data pertaining to a current activity of the user, an insulin intake data stream, an electrocardiography (ECG) data stream, a photoplethysmography data stream, and a data stream comprising data inputted by the user.

19. The system of claim 16, wherein the one or more applications stored in the non-transitory memory that, when executed by the processor:
select a subset of the one or more features;
generate a prediction of whether or not the hypoglycemic event or the hyperglycemic event will occur by the prediction model based on the selected subset.

20. The system of claim 16, wherein the one or more applications stored in the non-transitory memory that, when executed by the processor:
generate a prediction of whether or not a hypoglycemic event or a hyperglycemic event will occur at any point in time within a predefined prediction time window by a prediction model based on the one or more features.
